(19) United States
(12) Patent Application Publication
(10) Pub. No.: US 2020/0046815 A1
(43) Pub. Date: Feb. 13, 2020

(54) MULTIPLE-VARIABLE DOSE REGIMEN FOR TREATING DIABETES

(71) Applicant: KAMADA LTD., Ness Ziona (IL)

(72) Inventors: Pnina STRAUSS, Mazkeret Batya (IL); Naveh TOV, Haifa (IL)

(21) Appl. No.: 16/341,363

(22) PCT Filed: Oct. 10, 2017
(86) PCT No.: PCT/IL2017/051132

§ 371 Date: Apr. 11, 2019

Related U.S. Application Data
(60) Provisional application No. 62/407,550, filed on Oct. 13, 2016.

(51) Int. Cl. A61K 38/57; A61P 3/10

(52) U.S. Cl. CPC A61K 38/57; A61K 9/0019; A61P 3/10

(57) ABSTRACT

The present invention relates to the field of preservation of functional pancreatic islet (beta-cells) and treatment of diabetes, providing improved dosage regimen of AAT administration to Type 1 Diabetes Mellitus (T1DM) patients, particularly to newly diagnosed T1DM patients. The improved dose regiment is a multiple variable dosage regimen, comprising and induction phase and a treatment phase.

### FIELD OF THE INVENTION

The present invention relates to the field of preservation of functional pancreatic islet (beta-cells) and treatment of diabetes, providing improved dosage regimen of AAT administration to Type 1 Diabetes Mellitus (T1DM) patients, particularly to newly diagnosed T1DM patients.

### BACKGROUND OF THE INVENTION

Diabetes mellitus is a family of disorders characterized by chronic hyperglycemia and the development of long-term vascular complications. This family of disorders includes Type 1 diabetes, Type 2 diabetes, gestational diabetes, and other types of diabetes. Type 1 Diabetes Mellitus (T1DM), formerly called juvenile diabetes, is typically diagnosed in children, teenagers or young adults. The disease is the result of an autoimmune process that progressively destroys the insulin-producing pancreatic beta cells of the islets of Langerhans. In most cases this process remains non-symptomatic and it is thus undetected until diabetes becomes clinically apparent. At that point the number of functional beta cells is insufficient to produce the amount of insulin that is required to maintain glucose homeostasis. The level of C-peptide in blood (also known as Insulin C-peptide) helps to detect and diagnose the point where hyperglycemia is detected and differentiate between Type 1 from Type 2 Diabetes. A person whose pancreas does not make any insulin (type 1 diabetes) has a low level of insulin and C-peptide while a person with type 2 diabetes can have a normal or high level of C-peptide.

Although T1DM is essentially an autoimmune T cell-mediated disease, inflammation plays a pivotal role in multiple aspects of the condition, including islet function and survival as well as immune cell activation and maturity. In newly diagnosed T1DM patients (also referred to as recent onset T1DM patients), the inflammatory chemokines CCL3, CCL4 and CXCL10 have been detected in serum samples. A longitudinal study that examined 256 patients with recent onset T1DM found a negative correlation between CCL3 and C-peptide levels. Another study analyzed cytokine profiles in peripheral blood mononuclear cells obtained from patients with recent onset T1DM in comparison with the profiles of individuals with diabetes and healthy controls. An inflammatory expression pattern characterized by interleukin-(IL)-1 cytokine family members was identified in the T1DM patients. This inflammatory signature pattern appears to have been present years before clinical onset in a number of patients that participated in this study. These data, together with the well established role of IL-1β in islet destruction as a single-cytokine provocateur of islet beta cell death (Mandrup-Poulsen T et al. 1986. Diabetologia 29(1):63-7), increases the likelihood of the rational that the Type 1 diabetics is directly connected to inflammation.

Type-1 diabetes is currently managed by administration of exogenous human recombinant insulin. However, although insulin administration is effective in controlling the blood glucose level, it cannot stop the ongoing autoimmune destruction of beta cells. Several reports evaluating the effect of cytotoxic or immune modulator drugs on the prevention of T1DM have shown delayed decline of C-peptide level. Islet transplantation followed by a degree of immunosuppression has also shown much promise. However, continuous immunosuppression has some disadvantages including numerous side effects and relapse of beta cell destruction when treatment is stopped.

AAT is a heavily glycosylated plasma protein of 52 kDa in size. AAT is produced by the liver and secreted into the circulation, and is also produced locally by lung epithelial cells. Circulating levels of AAT increase during acute phase response. This increase is due to the presence of IL-1 and IL-6 responsive elements inside the promoter region of the AAT encoding gene. AAT functions as a serine protease inhibitor that primarily targets elastase, trypsin and proteinase-3, three immune cell-derived enzymes that are involved in protease-activated receptor (PAR) activation and the onset and progression of inflammation (Vergnolle N. 2009. Pharmacol Ther 123(3):292-309). Important pro-inflammatory mediators such as IL-1β, IL-6, IL-8 and TNFα are enhanced by these serine proteases and hence blocked by serine protease inhibitors, in particular by AAT (Pott G B et al. 2009. J Leukoc Biol. 85(5):886-95). Moreover, AAT induces the production and release of anti-inflammatory mediators such as IL-10 and IL-1-receptor antagonist (IL-1Ra) (Lewis E C et al. 2008. Proc Natl Acad Sci USA. 105(42):16236-41). In AAT deficiency, individuals are found to be at risk for lung alveolar tissue over-digestion by elastase; systemic inflammation is evident by a higher incidence of vasculitis.

Multiple studies over the past two decades and some earlier (as far back as the year 1967), assessed AAT levels and activity in diabetic individuals, from young and adolescent diabetics to pregnant women with diabetes, and found that the circulating levels of AAT are basically unaltered or even elevated by the disease; however, in a striking majority of cases, the activity of AAT was severely compromised by non-enzymatic glycations, supporting the conclusion that the inhibitory capacity of serum protease is reduced in T1DM. Evidence supporting this conclusion was found in a study of forty-nine children (24 girls and 25 boys) with T1DM and 24 non-diabetic children (13 girls and 11 boys). Means, medians and trypsin inhibitory capacity (TIC) of serum AAT were lower in diabetic children compared to healthy children (Lisowska-Myjak B et al. 2006. Acta Diabetol 43(4):88-92).

The glycation process of AAT and other proteins occurs primarily in the producing liver cells, and is time-dependent. The process requires approximately one week, or less, if the concentrations of glucose are significantly elevated. Indeed, as proven experimentally, one week after artificial induction of hyperglycemia in mice, circulating AAT is found to be inactivated (Hernandez-Espinosa D et al. 2009. Thromb Res. 124(4):483-489).

The use of AAT for reducing or preventing rejection of a cellular transplant and for treating diabetes has been suggested. For example, U.S. Pat. No. 8,071,551 discloses a method of treating an animal suffering a disease characterized by excessive apoptosis by administering a therapeutically effective amount of at least one serine protease inhibitor, and particularly a method for treating diabetes consisting of administering a therapeutically effective amount of alpha-1-antitrypsin (AAT) polypeptide.

U.S. Patent Applications Publications Nos. 2009/0118162 and 2009/0220518 disclose methods of treating, reducing or preventing rejection of a cellular transplant and/or side effects associated with transplantation by administering serine protease inhibitors, particularly AAT. In specific embodiments, the cellular transplant is pancreatic islets, and a method of treating diabetes that comprises pancreatic islet cell transplantation and administration of AAT is disclosed.

Several clinical trials address the potential benefit of AAT therapy to individuals with normal AAT production (i.e. not defined as AAT deficient subjects), including islet and lung transplantation, T1DM, graft-versus-host disease, acute myocardial infarction, and cystic fibrosis. The initial dosing plan in many of these trails, particularly in T1DM trails, was taken from the long-standing protocols of AAT augmentation therapy for AAT-deficient patients, e.g., weekly infusions of 60-80 mg/Kg plasma-derived affinity-purified human AAT. For example, Gottlieb et al, (Gottlieb P A met al., 2014. J Clin Endocrinol Metab 99:E1418-E1426) describe a trails with 12 participants 12-39 years old newly diagnosed for T1DM that received a fixed dose of 80 mg/KgBW by 8 consecutive weekly infusion. 18-months follow up showed improved circulating c-peptide in 5 of the patients. Recently, the Applicant of the present invention and others have designed clinical trials for assessing the safety and efficacy of several doses of AAT for treating T1DM (NCT02005848 and NCT02093221, respectively).

However, the timing, dosage and duration of AAT treatment required for the preservation of pancreatic islet function and/or treating newly diagnosed T1DM patients cannot be simply extrapolated from those found to be effective in treating the genetic AAT deficiency and the disorders associated thereto, particularly emphysema. First, T1DM is an immune disorder, while AAT deficiency and emphysema are not. Second, AAT deficiency is an ongoing constant shortage in AAT production, while the process of the onset of T1DM is not completely understood. Continuous release of AAT into the circulation as achieved by gene delivery of a human AAT expressing plasmid to mice, despite markedly lower levels of circulating AAT compared with those attained by infusion based approaches, resulted in some protection of transplanted islets (Shahaf G. et al., 2011. Mol Med 17:1000-1011).

There is an unmet need for an effective treatment of newly diagnosed T1DM patients that will preserve functional islet beta cells and prevent their destructions for a prolonged period of time, so as to maintain normal glycemic levels and reduce the need for exogenous insulin.

### SUMMARY OF THE INVENTION

The present invention provides a method for treating T1DM patients, particularly recent onset T1DM patients by employing a variable multiple-dose regimen of AAT administration.

The present invention is based in part on the unexpected discovery that administering AAT in a multiple-dose regimen comprising an induction phase followed by a treatment phase resulted in prolonged survival of transplanted pancreatic islets in model mice as was measured by the blood glucose level. The induction phase contained one administration of AAT at a high dose or several low doses at high administration frequency that accumulated to a high dose and the treatment phase contained repeated applications of AAT doses at lower frequency and/or doses.

Without wishing to be bound by any particular theory or mechanism of action this phenomenon may be attributed to the higher dose of AAT provided in the induction phase protecting the islet mass from rejection while the continuous, typically lower doses of AAT during the treatment phase provides immunomodulatory and protective anti-inflammatory environment, resulting in long term survival and function of the transplanted islets. Similarly, in newly diagnosed T1DM patient, the induction phase may attribute to the preservation of the functional islet beta cell mass while the treatment phase may contribute to preserving their function by reducing inflammatory processes. Particularly, the induction phase may contain one administration of AAT at a high dose or repeated administration of doses that accumulate to a high dose and the treatment phase may contain repeated administrations AAT doses wherein each dose is lower compared to the accumulated dose of the induction phase. Then intervals of AAT administration in the induction and treatment phase may contain prolong intervals of no administration. The frequency of administration during the induction and treatment phase can be the same or different, typically with lower administration frequency during the treatment phase.

According to one aspect, the present invention provides a method for treating Type 1 Diabetes Mellitus (T1DM) comprising administering to a subject in need thereof AAT in a multiple variable dosage regimen.

According to certain embodiments, the subject is newly diagnosed for T1DM. According to other embodiments, the newly diagnosed subject has C peptide levels of at least >0.2 ng/ml. According to certain embodiments, the subject is a pre-pubertal child, typically at or below 9 years of age. According to other embodiments, the subject is pre-pubertal adolescent at the age of 9-13 years. According to yet other embodiments, the subject is an adolescent at the age of 13-18 years. According to additional embodiments, the subject is an adult over 18 years old. According to additional embodiments, the subject is at the age of 12-25 years.

According to certain embodiments, the multiple variable dosage regimen comprises an induction phase and a treatment phase, wherein the induction phase comprise administrating AAT at a higher cumulative AAT dose and/or a higher frequency of AAT administration compared to the treatment phase.

According to certain embodiments, the induction phase comprises administering AAT at a cumulative dose that is higher than a single AAT dose administered during the treatment phase. The higher induction cumulative dose can be the result of a higher dose amount, higher administration frequency or a combination thereof. The amount of the AAT cumulative dose in the induction phase depends on the age, gender and the T1DM condition to be treated.

According to certain embodiments, the induction phase comprises administering AAT at a total cumulative amount in the range of from about 200 mg/Kg body weight (BW) to about 1,500 mg/KgBW. According to other embodiments, the AAT induction phase comprises administering AAT at a total cumulative amount in the range of from about 400 mg/KgBW to about 1,000 mg/KgBW. According to certain exemplary embodiments, the total cumulative amount of AAT during the induction phase is selected from the group consisting of 200, 360, 400, 480, 720, 960, 1000, 1440 and 1500 mg/KgBW. Each possibility represents a separate embodiment of the present invention.

According to some embodiments, the AAT induction phase comprises single dose administration of the total cumulative amount of AAT. According to these embodiments, the induction phase comprises from 1-14 days. According to certain exemplary embodiments, the induction phase comprises 7 days and the AAT is administered at the first day.

According to other embodiments, the AAT induction phase comprises multiple administrations of multiple portion doses to reach the total cumulative dose of AAT. According to certain embodiments, each portion dose comprises from about 40 mg AAT/KgBW to about 240 mg AAT/KgBW. According to other embodiments, each portion dose comprises 40, 60, 80, 120 or 240 mg AAT/KgBW. Each possibility represents a separate embodiment of the present invention.

According to the embodiments wherein AAT is repeatedly administered, the induction phase is in the range of from 1 week to 20 weeks. According to yet additional embodiments, the induction phase is in the range of from 3 week to 12 weeks. According to certain exemplary embodiments, the induction phase comprises 3 weeks. According to certain additional exemplary embodiments, the induction phase comprises 12 weeks.

According to certain embodiments, the induction phase comprises administering AAT in multiple portion doses at intervals of from 2-4 days to two weeks. According to certain embodiments, the interval between AAT portion dose administrations is constant. According to other embodiments, the interval between AAT portion dose administrations is variable. According to certain embodiments, the portion doses contain the same AAT amount. According to other embodiments, the portion doses contain variable AAT amounts.

According to certain exemplary embodiments, the AAT portion dose is administered once a week during the entire induction phase. According to certain exemplary embodiments, the AAT amount in each of the portion doses is constant during the entire induction phase.

The total cumulative dose of AAT to be administered during the treatment phase is variable and depends on the age, gender and the T1DM condition to be treated. According to typical embodiments, the treatment phase comprises multiple administrations of portion doses.

According to certain embodiments, the portion doses to be administered during the treatment phase comprise constant amount of AAT. According to other embodiments, the portion doses to be administered during the treatment phase comprise variable amount of AAT. According to certain exemplary embodiments, the amount of AAT in the treatment dose portion is descending from the first portion dose administered to the last portion dose administered.

According to certain embodiments, the AAT amount in the portion doses to be administered during the treatment phase is in the range of from 0 mg/KgBW to about 120 mg/KgBW. According to other embodiments, the AAT amount in the portion doses be administered during the treatment phase is in the range of from about 15 mg/KgBW to about 90 mg/KgBW. According to certain exemplary embodiments, the AAT amount in the portion doses to be administered during the treatment phase is selected from the group consisting of 15, 30, 40, 60, 80, 90 and 120 mg/KgBW and any combination thereof. Each possibility represents a separate embodiment of the present invention.

According to some embodiments, the treatment phase is in the range of from 6 weeks to 74 weeks. According to other embodiments, the treatment phase is in the range of from 8 weeks to 44 weeks. According to yet additional embodiments, the treatment phase is in the range of from 6 weeks to 16 weeks. According to certain exemplary embodiments, the duration of the treatment phase is selected from the group consisting of 6, 8, 16, 44 and 74 weeks. Each possibility represents a separate embodiment of the present invention

According to some embodiments, the AAT is administered at constant intervals during the entire length of the treatment phase. According to other embodiments, the AAT is administered at varying intervals during the treatment phase. According to certain exemplary embodiments, the AAT is administered at a constant treatment dose of 15, 40, 60, 80, 120 mg/Kg body weight. Each possibility represents a separate embodiment of the present invention.

According to certain exemplary embodiments, the method of the present invention comprises administering to a newly diagnosed T1DM subject AAT in a multiple variable dosage regimen, wherein the variable dosage regimen comprises an induction phase comprising administration a cumulative AAT dose of 120-960 mg AAT/KgBW and a treatment phase comprising administering at least two AAT doses, each dose comprising 0-120 mg/KgBW. According to additional exemplary embodiments, the cumulative AAT dose of the induction phase is administered in multiple dose portions. According to theses embodiments, the dose portions are administered 6-12 times at intervals of one week. According to further exemplary embodiments, the treatment phase comprises administering 2-4 AAT doses at intervals of 1-4 weeks.

According to additional exemplary embodiments, the present invention provides a method for treating T1DM comprising administering to a newly diagnosed T1DM subject AAT in a multiple dosage regimen, wherein the multiple-dosage regimen comprises: (a) an induction phase, the induction phase comprises administering 12 portion doses each of 60 or 120 mg AAT/KgBW at intervals of one week; (b) a treatment phase, the treatment phase comprising (i) administering four portion doses each of 60 or 120 mg AAT/KgBW at intervals of two weeks; (ii) no AAT administration for 26 weeks; (ii) administering 4 treatment portion doses each of 60 or 120 mg AAT/KgBW at intervals of two weeks; (iii) no AAT administration for 24 weeks; and (iv) administering 6 treatment portion doses each of 60 or 120 mg AAT/KgBW at intervals of one week; and a possibility (v) administering 6 treatment portion doses each of 60 or 120 mg AAT/KgBW at intervals of one week; wherein steps (a)-(b) and (i)-(v) are performed in a sequential order.

According to yet additional exemplary embodiments, the present invention provides a method for treating T1DM comprising administering to a newly diagnosed T1DM subject AAT in a multiple dosage regimen, wherein the multiple-dosage regimen comprises: (a) an induction phase, the induction phase comprises administering 3 doses each of 120 mg AAT/KgBW at intervals of one week; (b) a treatment phase, the treatment phase comprising (i) administering two doses each of 60 mg AAT/KgBW at intervals of two weeks; (ii) administering two doses each of 30 mg AAT/KgBW at intervals of two weeks; and (iii) administering 3 doses each of 15 mg AAT/KgBW at intervals of two weeks; wherein steps (a)-(b) and (i)-(iii) are performed in a sequential order.

According to further exemplary embodiments, the present invention provides a method for treating T1DM comprising administering to a newly diagnosed T1DM subject AAT in a multiple dosage regimen, wherein the multiple-dosage regimen comprises: (a) an induction phase, the induction phase comprises administering 12 doses each of 40, 60 or 80 mg AAT/KgBW at intervals of one week; (b) a treatment phase, the treatment phase comprising (i) administering 4 treatment portion doses each of 40, 60 or 80 mg AAT/KgBW at intervals of two weeks; and (ii) administering two treatment portion doses each of 40, 60 or 80 mg AAT/KgBW at intervals of four weeks; wherein steps (a)-(b) and (i)-(ii) are performed in a sequential order.

According to certain embodiments, the subject is human.

Any route of administration as is known in the art to be suitable for AAT administration can be used according to the teachings of the present invention. According to certain embodiments, the AAT is administered parenteraly. According to certain exemplary embodiments, the AAT is administered intravenously (i.v.). According to other embodiments, the AAT is administered by subcutaneous administration. The AAT is typically administered within a pharmaceutical composition formulated to complement with the route of administration.

According to another aspect, the present invention provides a method for prolonging cellular implant survival in a subject undergoing cellular implantation, comprising administering to the subject AAT in a multiple variable regimen, thereby prolonging the cellular implant survival.

According to certain embodiments, the multiple variable dosage regimen comprises an induction phase and a treatment phase, as described hereinabove.

According to certain embodiment, the cellular implant is in a form of a complete organ. According to other embodiments, the cellular implant is in a form of an organ part. According to yet additional embodiments, the implant is in a form of isolated cells or tissue. According to certain exemplary embodiments, the transplant is selected from the group consisting of a lung, a kidney, a cornea, pancreatic islets, a skin or any part thereof.

According to certain exemplary embodiments, the cellular implant is pancreatic islet cells. According to these embodiments, the subject is at risk to develop or has T1DM or Type 2 Diabetes Mellitus. According to additional embodiments, the subject is newly diagnosed for T1DM.

According to certain embodiments, the subject is human.

According to yet additional aspect, the present invention provides a kit for the treatment of T1DM comprising: (a) at least one portion of a pharmaceutical composition comprising an induction dose of AAT; (b) a plurality of portions of a pharmaceutical composition each comprising a treatment dose of AAT; and (c) instructions for administration of the induction dose of the AAT within an induction phase and of the treatment dose of the AAT within a treatment phase.

The AAT amount in each of the portions of the pharmaceutical composition, including the induction doses and the treatment doses are as described hereinabove. The instructions for administering the pharmaceutical portions comprising the induction and treatment doses of AAT include the administration regimens described hereinabove.

Other objects, features and advantages of the present invention will become clear from the following description and drawings.

### BRIEF DESCRIPTION OF THE FIGURES

FIG. 1 shows the effect of AAT on pancreatic islet graft survival at the standard 60 mg/Kg BW (n=10) compared to a half does of 30 mg/KgBW (n=10).

FIG. 2 shows the effect of AAT on pancreatic islet graft survival at the standard 60 mg/Kg BW (n=10) compared to the higher doses of 120 mg/KgBW (n=7) and 240 mg/KgBW (n=6).

FIG. 3 shows the effect of AAT on pancreatic islet graft survival at an extreme high dose of 240 mg/Kg BW (n=6) compared to an extreme low dose of 15 mg/KgBW (n=3).

FIG. 4 shows the effect of AAT administration at multiple fixed AAT doses on hAAT serum concentration of the receiving mice and on islet graft survival. FIG. 4A: Mice (n=3-5) were treated with 60 mg/kg i.p AAT as indicated (arrow). FIG. 4B: Mice were treated with 30 mg/kg (left) or 20 mg/kg (right) AAT i. p. at indicated time points (arrow; n=3 or 4 in each group). FIG. 4C: Islet graft survival curve. Groups include control (CT); the standard 60 mg/KgBW (n =10); ×3 of 20 mg/kg i.p (n=3) and ×2 of 30 mg/Kg i.p (n=6).

FIG. 5 shows the effect of AAT on pancreatic islet graft survival by administering AAT at variable doses. AAT was administered at the standard 60 mg/KgBW (n=10); at a high dose of 240 mg/KgBW (n=6) and at variable dose starting at 240- and descending to 60 mg/Kg BW (n=7).

FIG. 6 shows the effect of Glassia®, a ready-to-use AAT solution on pancreatic islet responses (FIG. 6A-B) and dendritic cell maturation (FIG. 6C) during inflammatory conditions. FIG. 6A: Islet viability and insulin release. FIG. 6B: Supernatant levels of nitric oxide, IL-6, MCP-1, and IL-10. FIG. 6C: Flow cytometry analysis of on bone-marrow derived dendritic cells. Representative results of three independent experiments. Mean±S.E.M., *P<0.05, **P<0.01.

FIG. 7 demonstrates Beta-cell function at 1 year endpoint subgroup analysis (12-25 years). The Primary efficacy endpoint was defined as the change from baseline in stimulated. C-peptide secretion, calculated as area under the curve (AUC), from a 2 hour mixed-meal tolerance test (MMTT). Peak secreted C-peptide is defined as Cmax.

FIGS. 8A-B demonstrate Glycemic control at 1 year subgroup analysis (12-25 years). Glycemic control was measured as % HbA1c, as efficacy was determined either as: average value at end of study (1 year) (FIG. 8A), or percent of patients who achieve HbA1c≤7% (FIG. 8B).

FIG. 9 demonstrates insulin requirement at 1 year subgroup analysis (12-25 years). Daily insulin dose were corrected by body weight (IU/kg). Data is shown as mean +/− SD.

### DETAILED DESCRIPTION OF THE INVENTION

The present invention discloses multiple-variable dosage method for treating diabetes, particularly in human subjects with newly diagnosed of Type 1 diabetes mellitus.

Definitions

The terms “diabetes” or “diabetic disorder” or “diabetes mellitus,” as used interchangeably herein, refer to a disease which is marked by elevated levels of sugar (glucose) in the blood. Diabetes can be caused by insufficient amount of insulin, resistance to insulin, or both. Diabetes includes the two most common types of the disorder, namely type I diabetes and type II diabetes, which both result from the body's inability to regulate insulin. Insulin is a hormone released by the pancreas in response to increased levels of glucose in the blood.

The terms “Type 1 diabetes”, “Type 1 diabetes mellitus” and “T1DM” are used herein interchangeably, referring to a chronic disease that occurs when the pancreas produces too little insulin to regulate blood sugar levels appropriately. Type 1 diabetes is also referred to as insulin-dependent diabetes mellitus, IDDM, and diabetes-type I. The disease often affects young children and thus also referred to as juvenile onset diabetes. Similar type 1 diabetes that affects older individuals is called “late onset” type 1 diabetes. Type 1 diabetes is the result of a progressive autoimmune destruction of the pancreatic β-cells with subsequent insulin deficiency.

As used herein, the term “Alpha-1 Antitrypsin” (AAT) refers to a glycoprotein that in nature is produced by the liver and lung epithelial cells and secreted into the circulatory system. AAT belongs to the Serine Proteinase Inhibitor (Serpin) family of proteolytic inhibitors. This glycoprotein consists of a single polypeptide chain containing one cysteine residue and 12-13% of the total molecular weight of carbohydrates. AAT has three N-glycosylation sites at asparagine residues 46, 83 and 247, which are occupied by mixtures of complex bi- and triantennary glycans. This gives rise to multiple AAT isoforms, having isoelectric point in the range of 4.0 to 5.0. The glycan monosaccharides include N-acetylglucosamine, mannose, galactose, fucose and sialic acid. AAT serves as a pseudo-substrate for elastase; elastase attacks the reactive center loop of the AAT molecule by cleaving the bond between methionine358 -serine359 residues to form an AAT-elastase complex. This complex is rapidly removed from the blood circulation. AAT is also referred to as “alpha-1 Proteinase Inhibitor” (API). The term “glycoprotein” as used herein refers to a protein or peptide covalently linked to a carbohydrate. The carbohydrate may be monomeric or composed of oligosaccharides. It is to be explicitly understood that any AAT as is or will be known in the art, including plasma-derived AAT and recombinant AAT can be used according to the teachings of the present invention.

The term “dosage” as used herein refers to the amount, frequency and duration of AAT which is given to a subject during a therapeutic period.

The term “dose” as used herein, refers to an amount of AAT which is given to a subject in a single administration.

The terms “multiple-variable dosage” and “multiple dosage” are used herein interchangeably and include different doses of AAT administration to a subject and/or variable frequency of administration of the AAT for therapeutic treatment. “Multiple dose regimen” or “multiple-variable dose regimen” describe a therapy schedule which is based on administering different amounts of AAT at various time points throughout the course of therapy. In one embodiment, the invention describes a multiple-variable dosage method of treatment comprising an induction phase and a treatment phase, wherein the AAT is administered at a total higher dose during the induction phase compared to the a single dose administered in the treatment phase.

The term “induction phase” as used herein refers to the first period of therapy comprising administration of AAT to a subject. During the induction phase, at least one induction dose of AAT is administered to a subject suffering from T1DM, particularly newly diagnosed T1DM subject.

The terms “induction dose” or “induction dose portion” refer to a dose of AAT administered during the induction phase of therapy.

The term “treatment phase” or “maintenance phase”, as used herein, refers to a period of therapy comprising administration of AAT to a subject in order to maintain a desired therapeutic effect. The treatment phase follows the induction phase. According to certain embodiments, the treatment phase starts at a pre-scheduled time point.

The terms “treatment dose” or “treatment dose portion” as used herein refer to a dose of AAT administered to a subject to maintain or continue a desired therapeutic effect during the treatment phase. A treatment dose is administered subsequent to the induction dose.

As used herein the term “about” refers to the designated value ±10%.

According to one aspect, the present invention provides a method for treating Type 1 Diabetes Mellitus (T1DM) comprising administering to a subject in need thereof AAT in a multiple variable dosage regimen.

According to certain embodiments, the subject is a newly diagnosed for T1DM.

According to certain embodiments, the multiple variable dosage regimen comprises an induction phase and a treatment phase, wherein the induction phase comprises administering AAT at a cumulative dose that is higher compared to each portion dose administered during the treatment phase.

Diabetes is often treated with diet, insulin dosages and various other medications. It is to be explicitly understood that the methods of the present invention can be employed together with at least one additional diabetes treatment. According to certain exemplary embodiments, the methods of the invention are employed together with administration of insulin. The multiple-variable dosage of AAT can be administered simultaneously or sequentially with the additional diabetes treatment.

The term “simultaneous administration,” as used herein, means that the AAT and the additional diabetes treatment are administered with a time separation of no more than about 15 minute(s), such as no more than about any of 10, 5, or 1 minutes.

The term “sequential administration” as used herein means that the AAT and the additional diabetes treatment are administered with a time separation of more than about 15 minutes, such as more than about any of 20, 30, 40, 50, 60 or more minutes. Either the AAT or the additional diabetes treatment may be administered first.

Evidence for islet protection by AAT has been previously described. With the induction of AAT, islets release less pro-inflammatory cytokines, primarily TNFα (Lewis E C et al. (2005) Proc Natl Acad Sci USA. 102(34):12153-8) which appears to be locked on cell membranes in a non-released form due to reduced TNFα-converting enzyme activity, and less of the toxic product nitric oxide.

The present invention is based in part on a model of implantation of pancreatic β-cells isolated from wild-type BALB/c into genetically engineered hAAT-Tg mice, background strain C57BL/6. Several groups of islet transplanted mice were divided into a series of novel AAT dose protocols. 60 mg/KgBW once every three days represents the human, once-weekly, standard protocol.

Islet transplantation can provide type-1 diabetes patients with tight glycemic control that can eliminate the need for exogenous insulin injections. In this procedure, isolated islets are introduced into the hepatic portal circulation of a diabetic patient. The immunosuppressive protocol used for islet transplantation excludes diabetogenic corticosteroids and therefore is void of anti-inflammatory activity. To date, islet loss in most transplant patients steadily progresses and results in a low graft survival rate of about 5 years.

Islets are particularly prone to injury during inflammatory conditions. Immediately after transplantation, viable islet mass rapidly decreases, regardless of allogeneic discrepancy. During this time, necrotic islet cells secrete injurious cytokines and chemokines while presenting allogeneic antigens to the host. Thus, grafted islets actively participate in the inflammatory burst and become activators, and targets, of endogenous macrophages.

The extent of inflammation and injury can determine the degree of antigen presentation and affects the expansion of allospecific effector cells. In addition, the favourable state of immune tolerance can be elaborated by a shift in balance between effector T cells and protective regulatory T (Treg) cells, a process which requires the uninterrupted activity of IL-2. By reducing the intensity of inflammation while allowing IL-2 activity one may provide optimal conditions for prolonged allograft survival.

In addition to its ability to inhibit serine proteases, AAT possesses anti-inflammatory properties. AAT has been described to prevent the demise of islet β-cells from normal mice, enabling insulin secretion in the presence of IL-β and IFNγ and reducing cytokine and chemokine secretion, previously described to be associated with the islet β-cell deterioration. It has been also described in model animals that AAT reduced the susceptibility of islets to inflammation and prolonged islet allograft survival. In addition, AAT allows for uninterrupted IL-2 activity.

As exemplified herein below the AAT effect can be divided into two distinct phases: a first phase of about 15-20 days in which the higher AAT doses are effective and a subsequent prolonged phase wherein lower AAT doses were more effective. Without wishing to be bound by any specific theory or mechanism of action, the higher initial dose protects the islet mass, and the lower AAT dose regulates the immune response of the transplanted mice.

The present invention thus discloses that neither phase is optimal on its own. Poor protection of islet mass might preclude graft survival in an otherwise protective immune environment; just as protection of mass is beneficial only for the critical first weeks but can be lost in an aggressive immune environment. Thus, the present invention now shows that administering the AAT in a multiple-variable dosage comprising an induction phase, comprising high administration frequency and/or cumulative dose followed by a treatment phase comprising lower AAT dosage (dose and/or frequency) results in improved survival of the transplanted β-cells in terms of viability and function.

Since both juvenile and late onset T1DM involve an autoimmune response directed against the insulin producing β-cells in the pancreatic islets, AAT can be used as a treatment for T1DM via the T-regulatory cells. In addition, the anti-inflammatory properties of AAT contribute to the protection of the pancreatic islet cells from the cytotoxic effects of pro-inflammatory cytokines and inflammatory mediators.

The present invention also shows the phenomenon of optimum-curve effect of AAT, with overdoses of AAT having negative effects. Without wishing to be bound by any specific theory or of action, AAT overdose may over-inhibit cytokine profile, wherein such inhibition has deleterious effects on β-cell function.

### Pharmaceutical Compositions

According to certain embodiments, AAT is administered in the form of a pharmaceutical composition. As used herein, the term “pharmaceutical composition” refers to a preparation of AAT with other chemical components such as pharmaceutically acceptable carriers and excipients. The purpose of a pharmaceutical composition is to facilitate administration of an active ingredient to an organism and enhance its stability and turnover.

Any available AAT as is known in the art, including plasma-derived AAT and recombinant AAT can be used according to the teachings of the present invention. According to certain exemplary embodiments, the AAT is produced by the method described in U.S. Pat. No. 7,879,800 to the Applicant of the present invention.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent or vehicle that does not cause significant irritation to an organism and does not abrogate the biological activity and properties of the administered compound. An adjuvant is included under these phrases. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions, isotonic buffers and physiological pH and aqueous dextrose and glycerol solutions can also be employed as liquid carriers, particularly for injectable solutions.

The pharmaceutical compositions of the invention can further comprise an excipient. Herein, the term “excipient” refers to an inert substance added to a pharmaceutical composition to further facilitate administration of an active ingredient. Suitable pharmaceutical excipients include starch, glucose, lactose, sucrose, trehalose, gelatin, malt, rice, flour, chalk, silica gel, sodium stearate, glycerol monostearate, talc, sodium chloride, dried skim milk, glycerol, propylene glycol, water, lipids, phospholipids, ethanol and the like. The composition, if desired, can also contain minor amounts of wetting or emulsifying agents, or pH buffering agents such as acetates, citrates or phosphates. Antibacterial agents such as benzyl alcohol or methyl parabens;

antioxidants such as ascorbic acid or sodium bisulfite; chelating agents such as ethylenediaminetetraacetic acid; and agents for the adjustment of tonicity such as sodium chloride or dextrose are also envisioned.

The pharmaceutical compositions of the present invention can be manufactured by processes well known in the art, e.g., by means of conventional mixing, dissolving, granulating, dragee-making, levigating, emulsifying, encapsulating, entrapping, spray drying or lyophilizing processes.

According to certain exemplary embodiments, pharmaceutical compositions, which contain AAT as an active ingredient, are prepared as injectable, either as liquid solutions or suspensions, however, solid forms, which can be suspended or solubilized prior to injection, can also be prepared. According to additional exemplary embodiments the AAT-containing pharmaceutical composition is formulated in a form suitable for inhalation. According to yet additional embodiments, the AAT-containing pharmaceutical composition is formulated in a form suitable for subcutaneous administration. Subcutaneous administration may be a preferred mode of administration, because administration of AAT at multiple low doses was shown to have a positive effect on islet protection. From the patient point of view multiple injections are not a favorable treatment, and thus it may be replaced by slow and/or controlled release subcutaneous administration. Any other forms of slow and/or controlled release are also explicitly encompassed within the scope of the present invention.

The compositions can also take the form of emulsions, tablets, capsules, gels, syrups, slurries, powders, creams, depots, sustained-release formulations and the like.

Methods of introduction of a pharmaceutical composition comprising AAT include, but are not limited to, intravenous, subcutaneous, intramuscular, intraperitoneal, oral, topical, intradermal, transdermal, intranasal, epidural, ophthalmic, vaginal and rectal routes. The pharmaceutical compositions can be administered by any convenient route, for example by infusion or bolus injection, by absorption through epithelial linings (e.g., oral mucosa, rectal and intestinal mucosa, etc.), and may be administered together with other therapeutically active agents. The administration may be localized, or may be systemic. Pulmonary administration can also be employed, e.g., by use of any type of inhaler or nebulizer.

Pharmaceutical compositions for use in accordance with the present invention may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, typically in physiologically compatible buffers such as Hank's solution, Ringer's solution, or physiological salt buffer. For transmucosal or transdermal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, traditional binders and carriers such as triglycerides, microcrystalline cellulose, gum tragacanth or gelatin.

According to certain exemplary embodiments, the AAT-containing pharmaceutical composition used according to the teachings of the present invention is a ready-to-use solution. According to further exemplary embodiments the AAT-containing pharmaceutical composition is marketed under the trade name Glassia®. The present invention is the first to show that a ready-to-use solution of AAT have a protective effect on primary islets.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Material and Methods

Mice: Seven week-old transgenic hAAT-heterozygous female mice, background strain C57BL/6, were used as graft recipient as previously described (Ashkenazi et al., 2013). Circulating levels of hAAT in these mice were determined to be below detection by a specific ELISA for human AAT (sensitivity 10 ng/ml in serum, Immunological Consultants Lab, Inc., Portland, Oreg.). These mice were used to avoid mouse anti-human antibody response. Wild-type BALB/c, were purchased from The Jackson Laboratory. Experiments were approved by the Ben-Gurion University Institutional Animal Care and Use Committee.

Islet Isolation: Islets were isolated from BALB/c mice on the day of transplantation, as described in Lewis et al. (Lewis E C, et al., 2008. Proc Natl Acad Sci U S A 105:16236-16241). Briefly, donor mice were anesthetized, and pancreata were inflated with collagenase (1 mg/ml, type XI, Sigma-Aldrich, Rehovot, Israel), excised, and incubated for 28-40 min at 37° C. Digested pancreata were vortexed and filtered through a 500 μm sieve and the pellet was washed in Hanks' balanced salt solution (HBSS) containing 0.5% bovine serum albumin (BSA) (Sigma-Aldrich). The pellet was resuspended in RPMI 1640 medium supplemented with 10% fetal calf serum (FCS), 50 units/ml penicillin, and 50 μg/ml streptomycin, all from Biologic industries, Beit-Haemek, Israel. Islets were collected on a 100-μm cell strainer (BD, Biosciences, Bedford, Mass.) and hand-picked under a stereomicroscope.

Pancreatic Islet Culture Experiments: Pancreatic islets were isolated as described hereinabove. For the in vitro studies, 35 islets per well in triplicates were stimulated with 5 ng/ml recombinant murine interferon (IFN)-γ and 5 ng/ml recombinant murine IL-1β (R&D Systems, Minneapolis, Minn.) in the absence or presence of 0.5 mg/ml human AAT (Glassia®, Kamada Ltd., Ness-Ziona, Israel). Forty-eight hours later, supernatants were collected for analysis by Q-Plex mouse cytokines chemiluminescence-based 8-p ELISA (Quansys Biosciences, Logan, Utah). Each cytokine was quantified by densitometry using Quansys Q-View software (Quansys Biosciences). Supernatant nitric oxide levels were evaluated by nitrite measurement using Griess reagent (Promega, Madison, Wis). Islet viability was determined by XTT assay, according to manufacturer's instructions (Sigma-Aldrich).

Islet Allograft Transplantation: Recipient mice were rendered hyperglycemic by single dose streptozotocin (STZ, i.p. 225 c mg/kg, Sigma-Aldrich), and 450 islets were grafted under the renal capsule, as described (Lewis et al., supra). Briefly, recipient mice were anesthetized, an abdominal-wall incision was made over the left kidney. Isolated islets were then released into the renal subcapsular space through a puncture in the capsule, which was rapidly sealed with 1-mm³ sterile absorbable gelatin sponge (Surgifoam, Ethicon, Somerville, N.J.).

hAAT treatment protocol: All in vivo hAAT treatments initiated 1 day before islet transplantation and were repeated every third day, unless otherwise stated. hAAT was administered i.p. at the dose indicated. Control hAAT-Tg mice received the same amount of human serum albumin (Abbott). Blood glucose levels were determined three times a week from tail blood by using a glucometer (Roche). Islet allograft rejection was defined as the day blood glucose exceeded 300 mg/dl after a period of at least 3 days of normoglycemia. Indefinite islet graft survival was defined as normoglycemia in grafted animals that lasted over 90 days.

hAAT distribution study: Serum from non-grafted hAAT-treated mice was collected using a designated microvette (Fisher Scientific, Waltham, Mass., USA). Circulating hAAT levels were detected using species-specific ELISA for human AAT (Immunological Consultants Lab, Inc.). Membrane-associated hAAT was determined by flow cytometry of thioglycolate-elicited peritoneal cell lavages using anti-hAAT-FITC (Bethyl Laboratories, Inc. Montgomery, Tex.) and anti-CD45-PE (eBioscience) antibodies. Peritoneal macrophages were pulsed with Glassia for indicated time points, lyzed and hAAT-content was depicted by western blot analysis using Goat anti-human AAT (Bethyl Laboratories, Inc.) and mouse anti-®-actin (MP Biomedicals, Santa Ana, Calif.) antibodies.

Generation of Bone Marrow Derived Dendritic Cells (BMDC): Dendritic cells were generated from bone marrow progenitors, as described elsewhere (Lewis et al., 2008, supra). Briefly, bone marrow was prepared from femurs and tibias of donor mice. Cells were seeded at 3×10³ cells per culture plate, in 10 ml RPMI 1640 medium supplemented with 10% fetal calf serum, 2 mM L-glutamine, 50 U/ml penicillin and 50 μg/ml streptomycin. 10 ng/ml recombinant granulocyte macrophage colony stimulating factor (GM-CSF, Prospec, Rehovot, Israel) were added to the medium, and the BMDC were used for the in vitro assays after 8 days.

DC Maturation Assay: Bone marrow-derived dendritic cells (BMDC) were stimulated with IFNγ and IL-1β (5 ng/ml each, Prospec), in the absence or presence of human AAT (0.5 mg/ml). Forty-eight hours later, supernatants were collected for cytokine and nitrite analysis. In the same manner, 24 hours after stimulation, cells were examined by flow cytometry, as described (Lewis et al., 2008, supra). The following antibodies were used for staining: anti-CD86-FITC, anti-MHC class II-PE and anti-CD11c-APC (all from eBioscience, San Diego, Calif.).

Statistics: Two-way ANOVA or Student's t-test was used to assess differences between groups. P<0.05 was considered statistically significant. Results are presented as mean±SEM.

### Example 1

### AAT Affects Islet Survival in an Optimum Curve

The effect of several doses of AAT on islet survival was examined. Each group of islet transplanted mice received a different AAT dose at the same administration frequency of once every three days as described hereinabove.

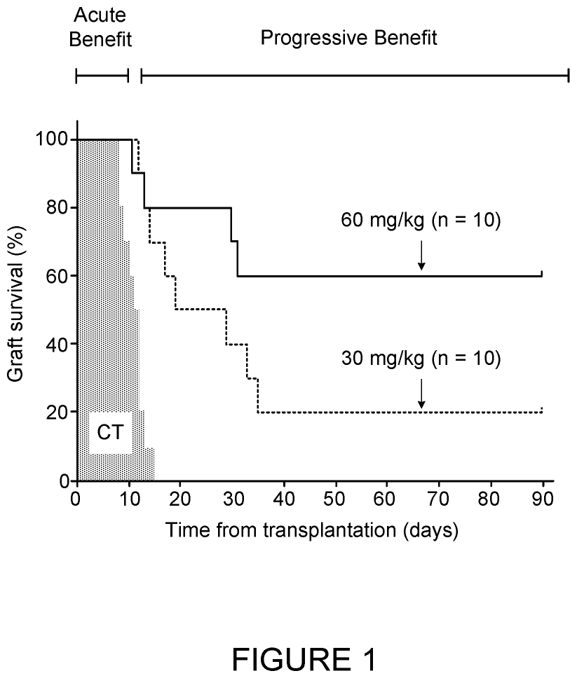

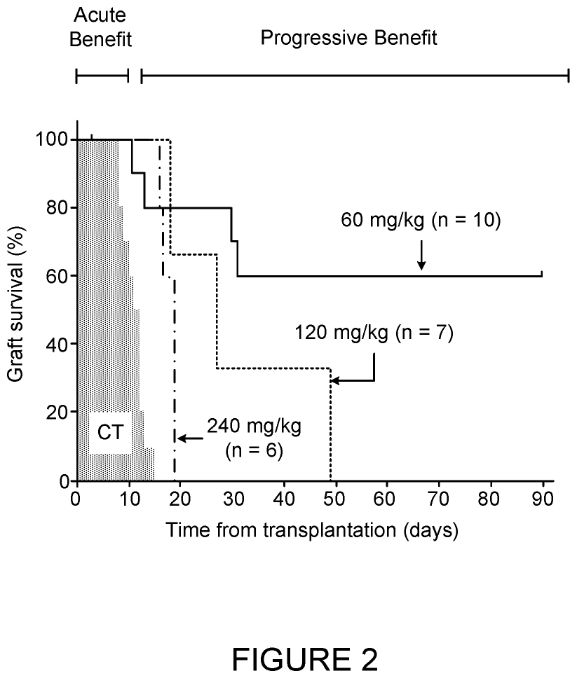

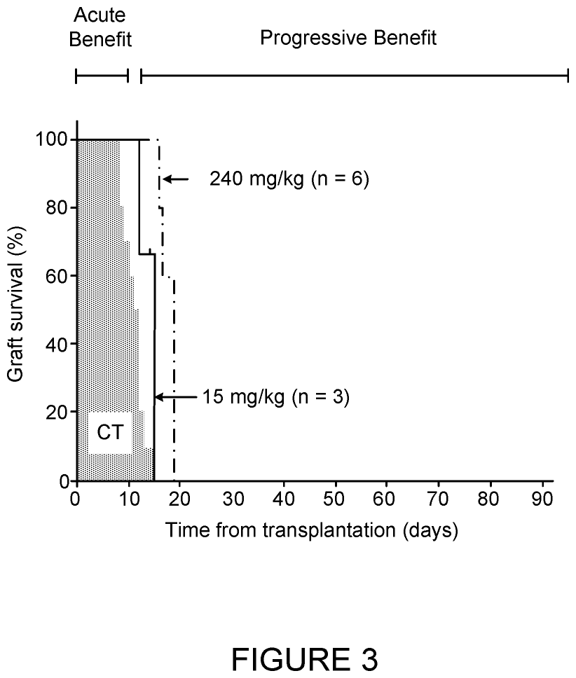

FIG. 1 shows the effect of the standard dose of 60 mg/KgBW compared to a half dose of 30 mg/KgBW. FIG. 2 shows that effect of higher doses of 120 and 240 mg/KgBW compared to the standard dose of 60 mg/KgBW. FIG. 3 shows the effect of two extreme high (240 mg/KgBW) and low (15 mg/KgBW) doses.

As shown, islet grafts in all untreated recipients (FIGS. 1-3, CT) failed to normalize blood glucose levels (indicating graft rejection) already before 20 days after transplantation. The standard 60 mg/KgBW resulted in prolonged graft survival, with 60% of the mice presenting indefinite graft survival of 90 days and more. Reducing the standard dose to a half dose of 30 mg/KgBW resulted in a decrease of the success rate, with only 20% of the mice presenting indefinite graft survival (FIG. 1).

Unexpectedly, the effect of the highest dose of 240 mg/KgBW was short term with a sharp decrease in graft survival at the 20^{th} day after transplantation (FIG. 2). This sharp decrease was similar to the effect of the lowest dose examined of 15 mg/KgBW (FIG. 3). However, the median point of graft rejection was delayed compared to the median time point reached for the control, untreated grafts, and also the earliest failing graft was delayed compared to the control (FIGS. 2-3). The dose of 120 mg/KgBW showed intermediate protection (FIG. 2). These results suggest an optimum curve of AAT protection of transplanted pancreatic islets as measured by the ability of the graft to keep normoglycaemia in the transplanted mice.

### Example 2

### Effect of Multiple Fixed AAT Dose on Islet Survival

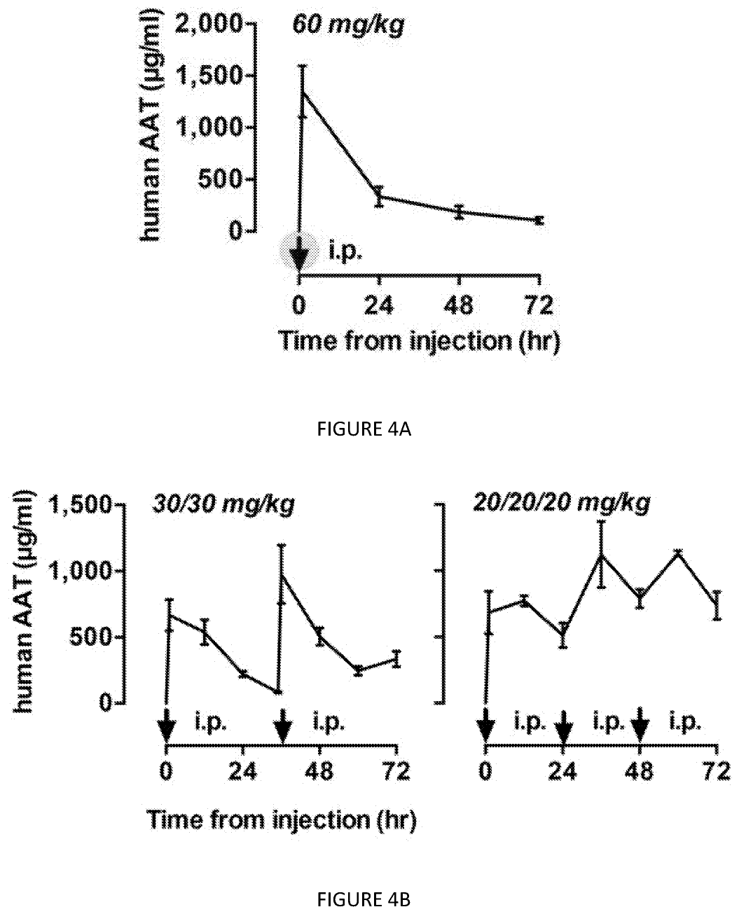

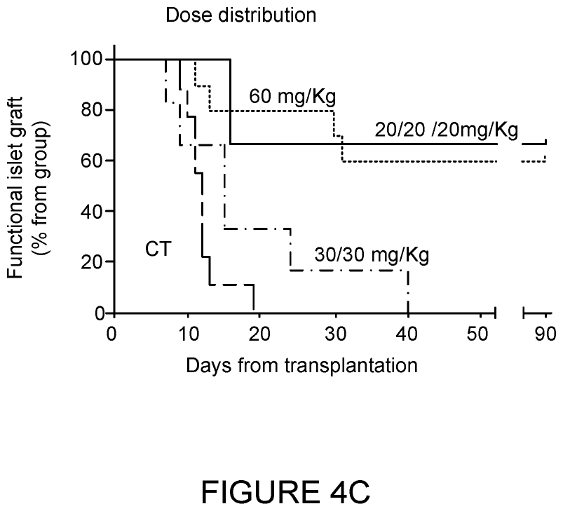

In view of the different protection afforded by high and low doses of AAT, it was examined whether replacing the fixed standard dose of 60 mg/KgBW with several lower doses administered at shorter intervals would be beneficial. Accordingly, mice received either single dose of 60 mg/KgBW. i.p or the same total amount of hAAT distributed as two or three separate rations. Tail blood was collected and serum hAAT levels were determined. As shown in FIG. 4A, the obtained mouse hAAT serum pharmacokinetics were similar to that reported in humans in that soon after administration circulating hAAT levels peaked (1,348.38±247.11 μg/ml), followed by dramatic reduction over the following 72 hours, reaching 103.98±31.67 μg g/ml. Dividing the standard dosage of 60 mg/KgBW to two or three portions given at prolonged time resulted in different circulating levels of hAAT. As shown in FIG. 4B, the 30 mg/Kg dose displayed the predicted circulating levels of 667.05±117.95 μg/ml and a kinetic that overlaps the full dose, and the additional dose of 30 mg/kg increased circulating hAAT levels to 976.1±221.03 μg/ml and a value of 69.32 μg/ml hAAT at the lowest measurement. However, AAT administration at 20 mg/KgBW every 24 hours over a period of 3 days resulted in a relatively stable range of concentrations, all above 226.65 μg/ml hAAT.

The ability of these dose distribution protocols to prolong islet graft survival was next examined. As shown in FIG. 4C, administration of 30 mg/KgBW AAT every 36 hours had a minor effect on graft survival, and all mice rejected the grafts by day 40 (n=6). Surprisingly, when mice were treated with daily 20 mg/KgBW, 66% displayed indefinite graft survival, comparable to the 60 mg/kg group (n=3).

### Example 3

### Effect of Multiple Variable AAT Dose on Islet Survival

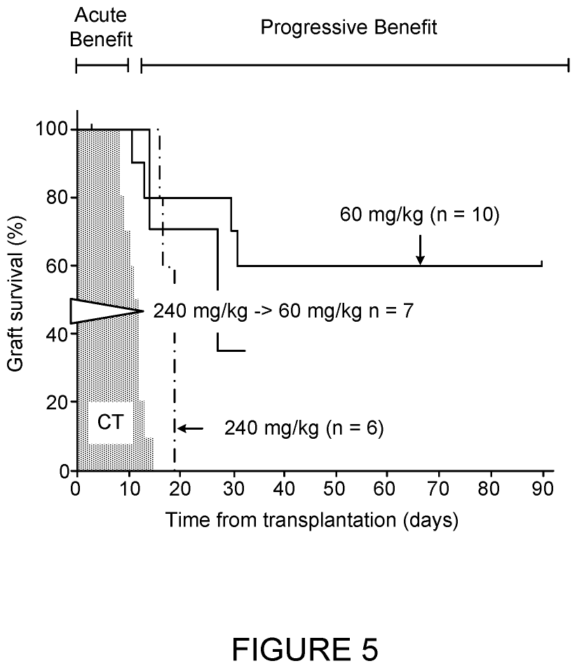

The result presented in FIG. 1-4 above revealed an unexpected phenomenon of two distinct phases of AAT effect: an initial phase of about 14-20 days from grafting in which AAT provides significant protection of islet survival and a subsequent more prolonged phase with varying degrees of islet protection. In the first phase, the higher doses of 120 and 240 mg Kg/BW provided better protection, while during the second phase the lower dose of 60 mg/Kg BW was significantly advantageous. Accordingly, a dynamic dosage range was attempted (FIG. 5). From the first injection of 240 mg/KgBW a dose of 60 mg/KgBW was reached by reducing each does in a gradual manner. The last injection was followed by one more 60 mg/KgBW injection and then ceased. As is shown in FIG. 5, reducing the dose gradually from 240 mg/KgBW provided an approved protection for the grafted islets compared to the administration of 240 mg/Kg dose. These results support the working hypothesis that administering a large amount of AAT at the acute phase followed by reduced amounts thereafter is beneficial to prolong graft survival, and similarly, to reserve the mass of active islets in newly diagnosed T1DM patient and their functionality thereafter.

### Example 4

### Maintenance of C-Peptide of T1D Newly Diagnosed Patients with AAT

The safety, tolerability and efficacy of intravenous AAT in the treatment of new onset Type 1 Diabetes Mellitus (T1DM) in young patients was studied through an open label, two-center study.

Methodology:

Subjects were assigned consecutively to three (3) dosage groups (8 subjects per group). The first group received a dose of 40 mg/Kg BW AAT throughout the study, the second group 60 mg/Kg BW and the third group 80 mg/Kg BW.

A total of up to 24 evaluable subjects with recently-diagnosed T1DM were enrolled and completed the study as planned.

Each study group completed the regime dose which included: an induction phase of 28 weeks consisting of 1 infusion per week for 12 weeks and then a treatment phase consisting of one infusion once every two weeks for 8 weeks and then one infusion every 4 weeks for 8 weeks.

Main inclusion criteria were:

Age 10-25 years (inclusive)

Diagnosed with T1DM within the previous 6 months

Level of C-peptide ≥0.2 pmol/mL during mixed meal tolerance test (MMTT, maximal level)

Positive for at least one diabetes-related autoantibody (except insulin autoantibody)

No significant abnormalities in serum hematology, serum chemistry, urinalysis and ECG according to the Investigator's judgment, taking into considerations the potential effects of the diabetic illness.

Outcome measures were safety, C-peptide reserve, insulin dose, and HbA1c.

Results:

Data were collected for 19 of the 24 participants (9 male, 10 female) who completed 12 infusions at the time of the interim analysis. Mean age of this cohort was 13.1 years (±2.7), and mean time elapsed from T1DM diagnosis to the first infusion was 84.3 days (±47.5).

No severe adverse events were reported during the study. Reported adverse events were mostly protein infusion-related; all were mild in intensity and resolved without sequelae.

HbA1c level, which is a commonly used endpoint for glycemic control is known to correlate with future disease complications. A statistically significant (p=<0.001) reduction in HbA1c relative to baseline was seen at all time intervals tested. More than 85% of subjects after the induction phase (12 weeks) had HbA1c levels lower than the clinically meaningful trough level of 7.5% considered being the maximal desirable trough level for T1D patients. More than 70% of subjects after the induction phase and the first part of the treatment phase (12 weeks+8 weeks) had HbA1c levels lower than the clinically meaningful trough level of 7.5% considered to be the maximal desirable trough level for T1D patients. More than 70% of subjects at the end of the treatment phase (week 28) had HbA1c levels lower than the clinically meaningful trough level of 7.5% considered being the maximal desirable trough level for T1D patients.

The mean reduction in HbA1c overall as compared to baseline was 14.8% at Week 5, 19.4% at Week 12, 16.3% at Week 20 and 16.2% at Week 28. At the Week-37 follow-up, the mean level of Hb1Ac was 15.7% lower than baseline. Reductions were seen in all dosage groups.

C-peptide level is a validated assessment of beta cell function. It is considered to be of major clinical significance during the first years following T1DM diagnosis, and is also considered as predictive of metabolic control and future disease complications. Within the study, all subjects had a Cmax of C-peptide that was equal to or greater than the clinically significant level of 0.2 pmol/L at Weeks 12 and 20. At Week 28, 20/22 subjects (90.9%) had levels of at least 0.2 pmol/L and at the 37 week follow-up, 78.3% had a Cmax of C-peptide that was at least 0.2 pmol/L.

End-of-study slope analysis of C-peptide [max] and C-peptide [AUC] revealed no significant changes from baseline. After 12-15 months from diagnosis, mean C-peptide [max] was 0.51±0.40pmol/mL vs. 0.69±0.42pmol/mL at baseline; AUC% decreased 23% from baseline (p=0.008).

Reduction in insulin consumption may also be indicative of better glycemic control and higher production of endogenous insulin. Interestingly, approximately half of all subjects showed a decrease in insulin dosage at Weeks 12, 20 and 28. At Week 37 follow-up, 8 out of 21 subjects for whom data was available (38.1%) showed a decrease in insulin dosage than at baseline (p=0.039). No statistically significant difference between dosage groups was revealed.

A statistically significant reduction in diabetes-related autoantibodies relative to baseline at the Week 37 follow-up was shown for the total study population. Reductions were seen in each treatment group, and overall the mean reduction in antibody level was 17.6% (p=0.003). Since the presence of such antibodies is indicative of beta-cell destruction, a reduction in antibody titer may indicate a slow-down in disease progression and remodulation of the immune system.

There were no evident trends in the occurrence of hypoglycemic events.

In conclusion, the improved metabolic control and beta-cell function after 12-15 months from diagnosis indicates that AAT administered in a variable dosage regimen exerts a protective effect on beta-cells, leading to a halt in disease progression and re-modulation of the autoimmune attack.

### Example 5

### Effect of Glassia®, a Ready-to Use AAT Solution on Primary Islet Function

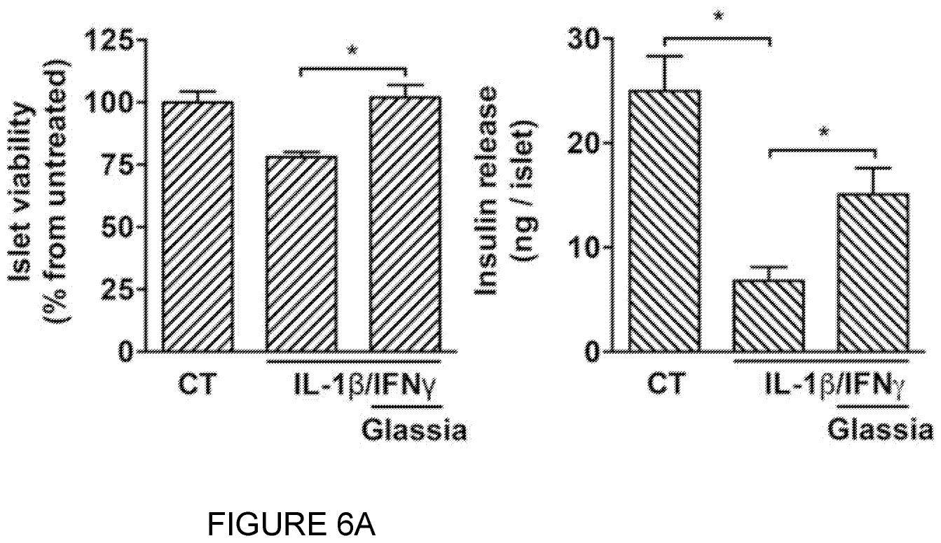

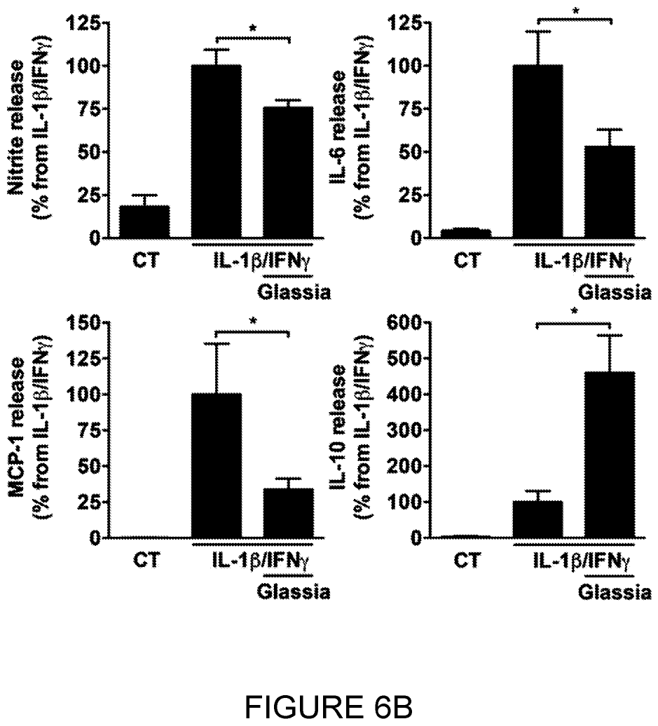

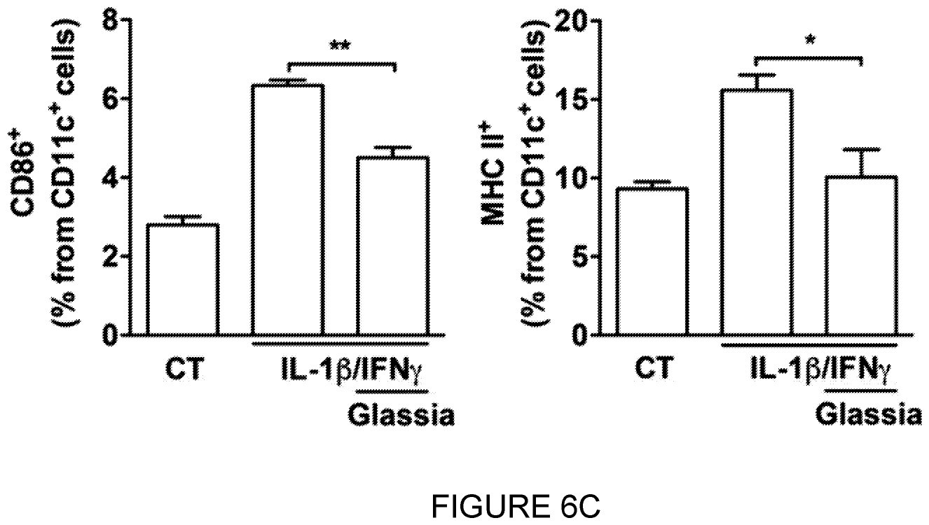

The present invention is exemplified by the use of Glassia®, a ready-to-use AAT solution manufactured by the Applicant of the present invention. To assess its effect on the function of inflamed islets, primary mouse islets were stimulated with interleukin (IL)-1β and interferon γ (IFNγ; 5 ng/ml each) in the absence or presence of Glassia® (0.5 mg/ml). As shown in FIG. 6A, 48 hours after stimulation islet viability was reduced, as expected, in the presence of IL-1β/IFNγ (78±0.01% viability compared with non-stimulated islets, albeit without reaching statistical significance). However, in the presence of Glassia®, islet viability was significantly improved and restored to near control levels. Accordingly, levels of insulin per islet released into the supernatants were significantly diminished by IL-1β/IFNγ-stimulation (a reduction of 3.67±0.05-fold from non-stimulated islets) and significantly increased in stimulated islets in the presence of Glassia (2.21±0.36-fold).

It was further examined whether the changes in the levels of inducible inflammatory mediators that are released by islet cells, namely, nitrite oxide, IL-6, and MCP-1, and of the anti-inflammatory mediator IL-10, are consistent with changes observed in previous reports. As shown in FIG. 6B, nitric oxide production levels were increased when IL-1β/IFNγ were added to the islets at 5.48±0.51-fold; when Glassia was added, a significant decline of nitric oxide levels were detected, as expected. Treatment with Glassia also reduced MCP-1 levels (33.8±0.07% from stimulated levels) and IL-6 levels (52.9±0.10% from stimulated levels). Although IL-10 levels increased in the presence of IL-1β/IFNγ, its levels further increased 4.6-fold in the presence of added Glassia. Next, the effect of Glassia on dendritic cell maturation was examined. Cultured bone marrow-derived dendritic cells were treated with IL-1β (5 ng/ml) and IFNγ (5 ng/ml) in the absence or presence of Glassia (0.5 mg/ml) and were then examined for surface activation markers by flow cytometry. As shown in FIG. 6C, stimulated dendritic cells exhibited a marked rise in the maturation markers CD86 and MHC class II; however, Glassia treatment resulted in diminished surface CD86 expression 51.4% from stimulated levels, mean), and surface MHC class II reached 13.160.11%, nearing control non-stimulated levels.

### Example 6

### A Phase II, Double-Blind, Randomized, Placebo-Controlled, Multicenter, Study Evaluating the Efficacy and Safety of Human, Alpha-1 Antitrypsin (AAT) in the Treatment of New Onset Type-1 Diabetes

A total of 70 patients recently diagnosed with Type-1 Diabetes were randomly assigned in a 1:1:1 ratio to either AAT intravenously (IV) 60 mg/kg body weight (Arm A), AAT IV 120 mg/kg body weight (Arm B) or placebo (Arm C).

Treatments and Follow-up are presented in Table 1:

| | |
|---|---|
| Treatment period | 52 weeks (or up to 82 weeks for those |
| | patients who completed treatment |
| | period 4) |
| Follow-up after last | 4 weeks (or up to 24 weeks for those |
| treatment day | patients who were already in follow-up |
| | period 2 or 3) |

Each patient underwent three treatment periods during 1 year. Those patients who completed treatment period 3 and had not yet entered follow-up period 2, were terminated the study and attended a study termination visit 4 weeks after the last dose of study treatment (Week 56). Patients who were already in follow-up period 2 were completed at least 4 weeks of follow-up before attending a study termination visit. For those patients who had already entered treatment period 4, they were completed the treatment period and attended a study termination visit 4 weeks after the last dose of study medication (Week 86). Patients who were already in follow-up period 2 were completed at least 4 weeks of follow-up before attending a study termination visit.

The duration of treatment for each individual patient varies depending on whether patient completed 52 weeks at the time of amendment implementation. Duration (including screening period) was approximately 60 weeks for those patients who completed treatment period 3 and approximately 90 weeks for those who had completed treatment period 4. For patients in the study the efficacy outcome data were covered up to Treatment Periods #1, 2 & 3 (i.e. up to week 52), and 4 weeks of follow-up (week 56).

Administration during the time-frame covered by efficacy outcome analysis was therefore consisting of:

1. From randomization, Twelve weekly dosing of AAT 60 or 120 mg/kg or Placebo

2. Four bi-weekly dosing of AAT 60 or 120 mg/kg or Placebo

3. Twenty six weeks of follow-up (no dosing)

4. Six weeks of weekly dosing of AAT 60 or 120 mg/kg or Placebo

Inclusion criteria were: Age 8-25 years, T1D diagnosed within 100 days, stimulated (MMTT) C-peptide ≥0.2 pmol/mL, and positive for at least 1 diabetes-related autoantibody (except insulin Ab).

Exclusion criteria were: recent vaccination with active/live virus, history of severe hypersensitivity to plasma products, evidence of ongoing viral infection with HAV, HCV, HBV, Parvovirus B19 or HIV-1,

Efficacy endpoints were defined at one year from study baseline. Primary efficacy endpoint: Beta-cell function as determined by MMTT-stimulated and C-peptide (AUC)

Secondary efficacy endpoints: Glycemic control as HbA1c level at one year from baseline and % of patients with HbA1c≤7.0%; Daily insulin dose as IU/kg; Safety and tolerability at end of follow-up: vital signs, physical examination, safety labs, SAEs and AEs.

Exploratory efficacy endpoints: Stimulated C-peptide (Cmax).

Study Population

Full Analysis Set (FAS): All randomized patients who received at least one dose of investigational treatment (AAT or placebo). Patients were included in the analysis according to the dose and Investigational Medicinal Product received, Patients who erroneously received a drug not according to randomization were counted according to the maximum dosage they received. A subject who received at least one intravenous AAT was assigned to the AAF treatment for the FAS population.

Subgroup Analysis

The younger patients present with a more “aggressive” progression of diabetes. Beta-cell function at diagnosis is usually lower and loss of function is faster, as determined by C-peptide secretion. For data analysis, patients were stratified according to age at randomization (based on definitions in SAP): children, age 8-11 years; adolescents, age 12-18 years and adults, age 19-25 years.

The efficacy outcome parameters of beta-cell function, glycemic control and insulin dose were analyzed in the adolescent and adult patients.

Patients were not randomized to treatment arm according to baseline parameters, resulting in an uneven distribution. There were relatively more children in the 120 mg/kg arm. Since there were very few adults in this study, adolescents and adults (12-25 years old) were pooled for comparison between treatment arms (Table 2). Treatment arms in the subgroup (12-25 years) were similar at baseline: age, time from diagnosis and baseline for beta-cell function (AUC and Cmax).

TABLE 2

| | Total | Children | Adolescents | Adults | Subgroup |
|---|---|---|---|---|---|
| Treatment | No. of | 8-11 | 12-18 | 19-25 | 12-25 |
| Arm | patients | years | years | years | years |
| AAT | 25 | 12 (48%) | 11 (44%) | 2 (8%) | 13 (52%) |
| 120 mg/kg | | | | | |
| AAT | 25 | 8 (32%) | 16 (64%) | 1 (4%) | 17 (68%) |
| 60 mg/kg | | | | | |
| Placebo | 19 | 7 (37%) | 8 (42) | 4 (21%) | 12 (63%) |

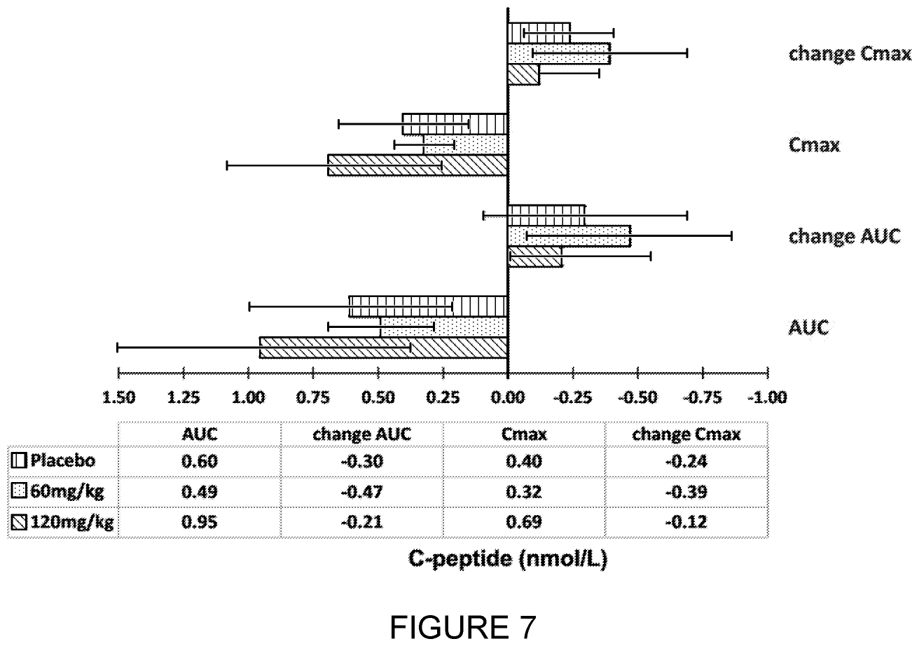

Beta-cell function at 1 year endpoint subgroup analysis (12-25 years) is presented in FIG. 7. The Primary efficacy endpoint was defined as the change from baseline in stimulated C-peptide secretion, calculated as area under the curve (AUC), from a 2 hour mixed-meal tolerance test (MMTT). Peak secreted. C-peptide is defined as Cmax.

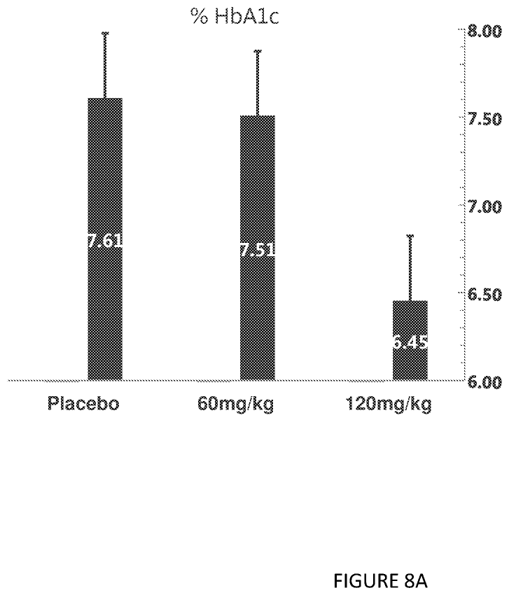

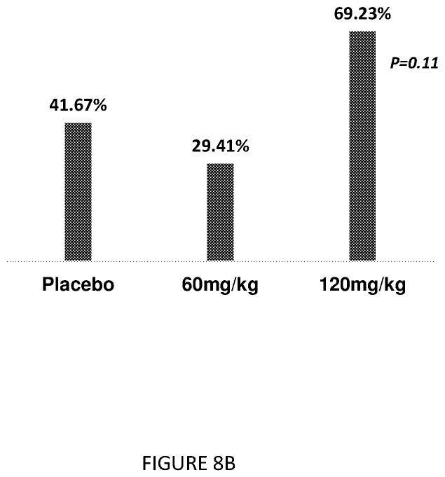

Glycemic control at 1 year subgroup analysis (12-25 years) is presented in FIGS. 8A-B. Glycemic control was measured as % HbA1c, as efficacy was determined either as: average value at end of study (1 year) ure 8A), or percent of patients who achieve HbA1c≤7% (FIG. 8B).

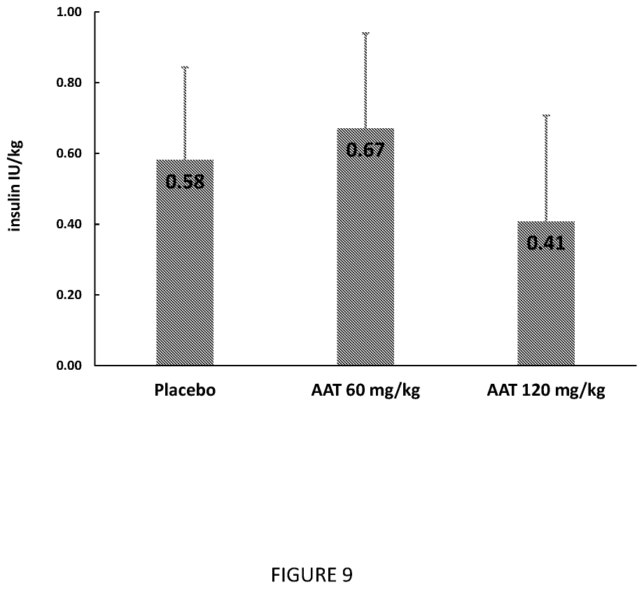

Insulin requirement at 1 year subgroup analysis (12-25 years) is presented in FIG. 9. Daily insulin dose were corrected by body weight (IU/kg). Data is shown as mean +/− SD.

The high dose arm of 120 mg/kg showed a better outcome of efficacy parameters at the 1-year end of study time point:

Higher beta-cell function, measured either as absolute values or as change from baseline, for stimulated C-peptide secretion AUC (0.95 vs. 0.60 nmol/L in Placebo, change −0.21 vs. −0.30 nmon in Placebo). Similar differences were obtained for the Cmax.

Better glycemic control was observed, whether expressed as average HbA1c or percent of patients who reach the target of ≤7% (70% vs. 42% in Placebo). Insulin data is partial; however the 120 mg/kg did not require more daily insulin to achieve a lower HbA1c level (0.41 IU/kg vs. 0.58 IU/kg in Placebo). This could fit with the observed higher beta-cell function.

The apparent better outcome for the 120 mg/kg treated patients is not due to higher beta-cell function at baseline, difference in age or time from diagnosis.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of treating Type 1 Diabetes Mellitus (T1DM) in a subject in need thereof, comprising administering to the subject alpha-1 antitrypsin (AAT) in a multiple variable dosage regimen comprising an induction phase followed by a treatment phase, wherein the cumulative dose of AAT administered during the induction phase is higher than the cumulative dose of AAT administered during the treatment phase.

2. The method of claim 1, wherein the subject is newly diagnosed for T1DM.

3. The method of claim 2, wherein the newly diagnosed subject has C peptide levels of at least >0.2 ng/ml.

4. The method of claim 1, wherein the subject is selected from the group consisting of a pre-pubertal child, a pre-pubertal adolescent, an adolescent and an adult.

5.-14. (canceled)

15. The method of claim 1, wherein each dose during the induction phase independently comprises from about 40 mg AAT/KgBW to about 240 mg AAT/KgBW.

16. The method of claim 15, wherein each dose during the induction phase independently comprises 40, 60, 80, 120 or 240 mg AAT/KgBW.

17. The method of claim 1, wherein the length of the induction phase is 3-12 weeks.

18. The method of claim 17, wherein the doses administered during the induction phase are administered at intervals of from 2 days to 2 weeks.

19. (canceled)

20. The method of claim 15, wherein each dose during the induction phase contains the same amount of AAT.

21. (canceled)

22. The method of claim 18, wherein the doses during the induction phase are administered at intervals of one week.

23. (canceled)

24. The method of claims 1, wherein each dose during the treatment phase comprises from 15 mg AAT/KgBW to about 120 mgAAT/KgBW.

25. The method of claim 24, wherein each dose during the treatment phase independently comprises 15, 30, 40, 60, 80, 90 or 120 mg AAT/KgBW.

26. The method of claim 24, wherein the each dose during the treatment phase contains the same amount of AAT.

27.-28. (canceled)

29. The method of claim 1, wherein the length the treatment phase is 6-74 weeks.

30.-33. (canceled)

34. The method of claim 52, wherein the induction phase comprises administering a cumulative AAT dose of 120-960 mg AAT/KgBW and the treatment phase comprises administering at least two AAT doses, each dose comprising 15-120 mg/KgBW.

35. (canceled)

36. The method of claim 34, wherein the induction phase comprises administering 6-12 times AAT doses at intervals of one week.

37. The method of claim 36, wherein the treatment phase comprises administering 2-4 AAT doses at intervals of 1-4 weeks.

38. The method of claim 52, wherein: (a) the induction phase comprises administering 12 doses each of 60 or 120 mg AAT/KgBW at intervals of one week; and, (b) the treatment phase comprises (i) administering four doses each of 60 or 120 mg AAT/KgBW at intervals of two weeks; (ii) no AAT administration for 26 weeks; (ii) administering 4 doses each of 60 or 120 mg AAT/KgBW at intervals of two weeks; (iii) no AAT administration for 24 weeks; and, (iv) administering 6 doses each of 60 or 120 mg AAT/KgBW at intervals of one week; and, optionally, (v) administering 6 doses each of 60 or 120 mg AAT/KgBW at intervals of one week; wherein steps (a)-(b) and (i)-(v) are performed in a sequential order.

39. The method of claim 2, wherein: (a) the induction phase comprises administering 3 doses each of 120 mg AAT/KgBW at intervals of one week; and, (b) the treatment phase comprises (i) administering two doses each of 60 mg AAT/KgBW at intervals of two weeks; (ii) administering two doses each of 30 mg AAT/KgBW at intervals of two weeks; and, (iii) administering 3 doses each of 15 mg AAT/KgBW at intervals of two weeks; wherein steps (a)-(b) and (i)-(iii) are performed in a sequential order.

40. The method of claim 2, wherein: (a) the induction phase comprises administering 12 doses each of 40, 60 or 80 mg AAT/KgBW at intervals of one week; and, (b) the treatment phase comprises (i) administering 4 doses each of 40, 60 or 80 mg AAT/KgBW at intervals of two weeks; and, (ii) administering two doses each of 40, 60 or 80 mg AAT/KgBW at intervals of four weeks; wherein steps (a)-(b) and (i)-(ii) are performed in a sequential order.

41.-42. (canceled)

43. The method of claim 1, wherein the AAT is selected from the group consisting of plasma-derived AAT and recombinant AAT.

44. (canceled)

45. The method of claim 1, wherein the AAT is administered via parenteral administration.

46. The method of claim 45, wherein the AAT is administered intravenously.

47. (canceled)

48. The method of claim 1, wherein the AAT is administered via inhalation.

49. A method of prolonging cellular implant survival in a subject in need thereof undergoing cellular implantation, comprising administering to the subject AAT in a multiple variable dosage regimen, thereby prolonging the cellular implant survival, the multiple variable dosage regimen comprising an induction phase followed by a treatment phase, wherein the cumulative dose of AAT administered during the induction phase is higher than the cumulative dose of AAT administered during the treatment phase.

50.-51. (canceled)
